(19) United States
(12) Patent Application Publication
(10) Pub. No.: US 2017/0281685 A1
(43) Pub. Date: Oct. 5, 2017

(54) NOBLE GAS AUGMENTATION OF REGENERATIVE CELL ACTIVITY

(71) Applicant: NOBLIS THERAPEUTICS, INC., Portland, OR (US)

(72) Inventors: Vlad Bogin, Portland, OR (US); Thomas Ichim, San Diego, CA (US)

(21) Appl. No.: 15/465,514

(22) Filed: Mar. 21, 2017

Related U.S. Application Data
(60) Provisional application No. 62/314,983, filed on Mar. 29, 2016.

(51) Int. Cl. A61K 35/28; C12N 5/0775

(52) U.S. Cl. CPC A61K 35/28; C12N 5/0668; C12N 2500/05

(57) ABSTRACT

The current invention discloses compositions of matter, therapeutic protocols, and combination therapies utilizing previously unknown activity of Xenon alone or in combination with other Noble gases to augment therapeutic activity of regenerative cells, in one embodiment said regenerative cells comprising of mesenchymal stem cells. In one embodiment the invention provides use of xenon-pretreatment of cells prior to in vivo application in order to allow for enhanced efficacy of said cells. In other embodiments xenon alone, or in combination with other gases is utilized to modulate regenerative activity of cells in vivo.

### CROSS-REFERENCE TO RELATED APPLICATIONS

The present invention claims priority to U.S. Provisional Application No. 62/314,983, filed Mar. 29, 2016, which is hereby incorporated in its entirety including all tables, figures, and claims

### FIELD OF THE INVENTION

The invention pertains to the area of regenerative medicine, more specifically, the invention pertains to the area of mesenchymal stem cell therapeutics, more specifically, the invention pertains to means of selecting stem cells possessing enhanced efficacy, furthermore the invention pertains to the area of stem cell efficacy markers.

### BACKGROUND

According to the definition by the U.S. Food and Drug Administration (FDA), somatic cell therapy (or cell therapy) is the prevention, treatment, cure, diagnosis, or mitigation of diseases or injuries in humans by the administration of autologous, allogeneic or xenogeneic cells that have been manipulated or altered ex vivo. Generally, said manipulation and alteration include the propagation, expansion, selection, and/or pharmacological treatment of the cells. The goal of cell therapy is to repair, replace or restore damaged tissues or organs. Cell therapy may provide extensive applications in modern medicine. For example, in Nov. 10, 2011, the U.S. FDA granted marketing approval to the New York Blood Center's allogeneic cord-blood product, HEMACORD, the first FDA-licensed hematopoietic progenitor cell therapy. HEMACORD is indicated for hematopoietic progenitor cell (HPC) transplantation procedures in patients with inherited, acquired, or myeloablative-treatment-related diseases that affect the hematopoietic system. Once the HPCs are infused into patients, the cells migrate to the bone marrow where they divide and mature. When the mature cells move into the bloodstream they can partially or fully restore the number and function of many blood cells, including immune function.

Mesenchymal stem cell therapeutics has entered the clinical arena in the treatment of various degenerative conditions including cardiovascular, neurological, and immunological. Regulatory approval of mesenchymal stem cell based products has been achieved in several jurisdictions, particularly of mesenchymal stem cells. Mesenchymal stem cells are classically defined as adherent cells possessing ability to differentiate into osteoblasts, adipocytes and chondrocytes and possessing the surface markers CD73, CD90, and CD105, while lacking the markers CD14, CD34, and CD45.

The invention teaches ways of augmenting MSC activity through exposure to Noble gas containing mixtures to enhance expression of HIF-1 alpha, which in turn augments efficacy of MSC based therapeutics.

### DESCRIPTION OF THE INVENTION

The invention teaches in vitro or in vivo treatment with Noble gas mixtures, in particular treatment with xenon, and/or xenon-argon, endows mesenchymal stem cells with “enhanced activity”, in particular upregulation of growth factor production, increased ability to migrate towards chemoattractant gradients, and augmented therapeutic activity.

“Enhanced MSC” refers to MSC or MSC-like cells that have been treated in vitro or in vivo with Noble gas mixtures to upregulated therapeutic activity.

Differentiation is the process by which an unspecialized (“uncommitted”) or less specialized cell acquires the features of a specialized cell, such as a nerve cell or a muscle cell, for example. A differentiated cell is one that has taken on a more specialized (“committed”) position within the lineage of a cell. The term committed, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. De-differentiation refers to the process by which a cell reverts to a less specialized (or committed) position within the lineage of a cell. As used herein, the lineage of a cell defines the heredity of the cell, i.e. which cells it came from and what cells it can give rise to. The lineage of a cell places the cell within a hereditary scheme of development and differentiation.

In a broad sense, a progenitor cell is a cell that has the capacity to create progeny that are more differentiated than itself, and yet retains the capacity to replenish the pool of progenitors. By that definition, stem cells themselves are also progenitor cells, as are the more immediate precursors to terminally differentiated cells. When referring to the cells of the present invention, as described in greater detail below, this broad definition of progenitor cell may be used. In a narrower sense, a progenitor cell is often defined as a cell that is intermediate in the differentiation pathway, i.e., it arises from a stem cell and is intermediate in the production of a mature cell type or subset of cell types. This type of progenitor cell is generally not able to self-renew. Accordingly, if this type of cell is referred to herein, it will be referred to as a non-renewing progenitor cell or as an intermediate progenitor or precursor cell.

As used herein, the phrase differentiates into a mesodermal, ectodermal or endodermal lineage refers to a cell that becomes committed to a specific mesodermal, ectodermal or endodermal lineage, respectively. Examples of cells that differentiate into a mesodermal lineage or give rise to specific mesodermal cells include, but are not limited to, cells that are adipogenic, chondrogenic, cardiogenic, dermatogenic, hematopoetic, hemangiogenic, myogenic, nephrogenic, urogenitogenic, osteogenic, pericardiogenic, or stromal. Examples of cells that differentiate into ectodermal lineage include, but are not limited to epidermal cells, neurogenic cells, and neurogliagenic cells. Examples of cells that differentiate into endodermal lineage include, but are not limited to, pleurigenic cells, hepatogenic cells, cells that give rise to the lining of the intestine, and cells that give rise to pancreogenic and splanchogenic cells.

The cells of the present invention are generally referred to as umbilicus-derived cells (or UDCs). They also may sometimes be referred to more generally herein as postpartum-derived cells or postpartum cells (PPDCs). In addition, the cells may be described as being stem or progenitor cells, the latter term being used in the broad sense. The term derived is used to indicate that the cells have been obtained from their biological source and grown or otherwise manipulated in vitro (e.g., cultured in a growth medium to expand the population and/or to produce a cell line). The in vitro manipulations of umbilical stem cells and the unique features of the umbilicus-derived cells of the present invention are described in detail below.

Various terms are used to describe cells in culture. Cell culture refers generally to cells taken from a living organism and grown under controlled condition (“in culture” or “cultured”). A primary cell culture is a culture of cells, tissues, or organs taken directly from an organism(s) before the first subculture. Cells are expanded in culture when they are placed in a growth medium under conditions that facilitate cell growth and/or division, resulting in a larger population of the cells. When cells are expanded in culture, the rate of cell proliferation is sometimes measured by the amount of time needed for the cells to double in number. This is referred to as doubling time.

A cell line is a population of cells formed by one or more subcultivations of a primary cell culture. Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but not limited to the seeding density, substrate, medium, growth conditions, and time between passaging.

A conditioned medium is a medium in which a specific cell or population of cells has been cultured, and then removed. When cells are cultured in a medium, they may secrete cellular factors that can provide trophic support to other cells. Such trophic factors include, but are not limited to hormones, cytokines, extracellular matrix (ECM), proteins, vesicles, antibodies, and granules. The medium containing the cellular factors is the conditioned medium.

Generally, a trophic factor is defined as a substance that promotes or at least supports, survival, growth, proliferation and/or maturation of a cell, or stimulates increased activity of a cell.

When referring to cultured vertebrate cells, the term senescence (also replicative senescence or cellular senescence) refers to a property attributable to finite cell cultures; namely, their inability to grow beyond a finite number of population doublings (sometimes referred to as Hayflick's limit). Although cellular senescence was first described using fibroblast-like cells, most normal human cell types that can be grown successfully in culture undergo cellular senescence. The in vitro lifespan of different cell types varies, but the maximum lifespan is typically fewer than 100 population doublings (this is the number of doublings for all the cells in the culture to become senescent and thus render the culture unable to divide). Senescence does not depend on chronological time, but rather is measured by the number of cell divisions, or population doublings, the culture has undergone. Thus, cells made quiescent by removing essential growth factors are able to resume growth and division when the growth factors are re-introduced, and thereafter carry out the same number of doublings as equivalent cells grown, continuously. Similarly, when cells are frozen in liquid nitrogen after various numbers of population doublings and then thawed and cultured, they undergo substantially the same number of doublings as cells maintained unfrozen in culture. Senescent cells are not dead or dying cells; they are actually resistant to programmed cell death (apoptosis), and have been maintained in their nondividing state for as long as three years. These cells are very much alive and metabolically active, but they do not divide. The nondividing state of senescent cells has not yet been found to be reversible by any biological, chemical, or viral agent.

As used herein, the term Growth Medium generally refers to a medium sufficient for the culturing of umbilicus-derived cells. In particular, one presently preferred medium for the culturing of the cells of the invention herein comprises Dulbecco's Modified Essential Media (also abbreviated DMEM herein). Particularly preferred is DMEM-low glucose (also DMEM-LG herein) (Invitrogen, Carlsbad, Calif.). The DMEM-low glucose is preferably supplemented with 15% (v/v) fetal bovine serum (e.g. defined fetal bovine serum, Hyclone, Logan Utah), antibiotics/antimycotics (preferably penicillin (100 Units/milliliter), streptomycin (100 milligrams/milliliter), and amphotericin B (0.25 micrograms/milliliter), (Invitrogen, Carlsbad, Calif.)), and 0.001% (v/v) 2-mercaptoethanol (Sigma, St. Louis Mo.). In some cases different growth media are used, or different supplementations are provided, and these are normally indicated in the text as supplementations to Growth Medium.

Also relating to the present invention, the term standard growth conditions, as used herein refers to culturing of cells at 37.degree. C., in a standard atmosphere comprising 5% CO.sub.2. Relative humidity is maintained at about 100%. While foregoing the conditions are useful for culturing, it is to be understood that such conditions are capable of being varied by the skilled artisan who will appreciate the options available in the art for culturing cells, for example, varying the temperature, CO.sub.2, relative humidity, oxygen, growth medium, and the like.

“Mesenchymal stem cell” or “MSC” in some embodiments refers to cells that are (1) adherent to plastic, (2) express CD73, CD90, and CD105 antigens, while being CD14, CD34, CD45, and HLA-DR negative, and (3) possess ability to differentiate to osteogenic, chondrogenic and adipogenic lineage. Other cells possessing mesenchymal-like properties are included within the definition of “mesenchymal stem cell”, with the condition that said cells possess at least one of the following: a) regenerative activity; b) production of growth factors; c) ability to induce a healing response, either directly, or through elicitation of endogenous host repair mechanisms. As used herein, “mesenchymal stromal cell” or ore mesenchymal stem cell can be used interchangeably. Said MSC can be derived from any tissue including, but not limited to, bone marrow, adipose tissue, amniotic fluid, endometrium, trophoblast-derived tissues, cord blood, Wharton jelly, placenta, amniotic tissue, derived from pluripotent stem cells, and tooth. In some definitions of “MSC”, said cells include cells that are CD34 positive upon initial isolation from tissue but are similar to cells described about phenotypically and functionally. As used herein, “MSC” may includes cells that are isolated from tissues using cell surface markers selected from the list comprised of NGF-R, PDGF-R, EGF-R, IGF-R, CD29, CD49a, CD56, CD63, CD73, CD105, CD106, CD140b, CD146, CD271, MSCA-1, SSEA4, STRO-1 and STRO-3 or any combination thereof, and satisfy the ISCT criteria either before or after expansion. Furthermore, as used herein, in some contexts, “MSC” includes cells described in the literature as bone marrow stromal stem cells (BMSSC), marrow-isolated adult multipotent inducible cells (MIAMI) cells, multipotent adult progenitor cells (MAPC), mesenchymal adult stem cells (MASCS), MultiStem®, Prochymal®, remestemcel-L, Mesenchymal Precursor Cells (MPCs), Dental Pulp Stem Cells (DPSCs), PLX cells, PLX-PAD, AlloStem®, Astrostem®, Ixmyelocel-T, MSC-NTF, NurOwn™, Stemedyne™-MSC, Stempeucel®, StempeucelCLI, StempeucelOA, HiQCell, Hearticellgram-AMI, Revascor®, Cardiorel®, Cartistem®, Pneumostem®, Promostem®, Homeo-GH, AC607, PDA001, SB623, CX601, AC607, Endometrial Regenerative Cells (ERC), adipose-derived stem and regenerative cells (ADRCs).

Oct-4 (oct-3 in humans) is a transcription factor expressed in the pregastrulation embryo, early cleavage stage embryo, cells of the inner cell mass of the blastocyst, and embryonic carcinoma (“EC”) cells (Nichols, J. et al. (1998) Cell 95: 379-91), and is down-regulated when cells are induced to differentiate. The oct-4 gene (oct-3 in humans) is transcribed into at least two splice variants in humans, oct-3A and oct-3B. The oct-3B splice variant is found in many differentiated cells whereas the oct-3A splice variant (also previously designated oct-3/4) is reported to be specific for the undifferentiated embryonic stem cell. See Shimozaki et al. (2003) Development 130: 2505-12. Expression of oct-3/4 plays an important role in determining early steps in embryogenesis and differentiation. Oct-3/4, in combination with rox-1, causes transcriptional activation of the Zn-finger protein rex-1, which is also required for maintaining ES cells in an undifferentiated state (Rosfjord, E. and Rizzino, A. (1997) Biochem Biophys Res Commun 203: 1795-802; Ben-Shushan, E. et al. (1998) Mol Cell Biol 18: 1866-78).

The term “neoplasm” generally denotes disorders involving the clonal proliferation of cells. Neoplasms may be benign, which is to say, not progressive and non-recurrent, and, if so, generally are not life-threatening. Neoplasms also may be malignant, which is to say, that they progressively get worse, spread, and, as a rule, are life threatening and often fatal.

Inflammatory conditions is an inclusive term and includes, for example: (1) tissue damage due to ischemia-reperfusion following acute myocardial infarction, aneurysm, stroke, hemorrhagic shock, crush injury, multiple organ failure, hypovolemic shock intestinal ischemia, spinal cord injury, and traumatic brain injury; (2) inflammatory disorders, e.g., burns, endotoxemia and septic shock, adult respiratory distress syndrome, cardiopulmonary bypass, hemodialysis; anaphylactic shock, severe asthma, angioedema, Crohn's disease, sickle cell anemia, poststreptococcal glomerulonephritis, membranous nephritis, and pancreatitis; (3) transplant rejection, e.g., hyperacute xenograft rejection; (4) pregnancy related diseases such as recurrent fetal loss and pre-eclampsia, and (5) adverse drug reactions, e.g., drug allergy, IL-2 induced vascular leakage syndrome and radiographic contrast media allergy. Complement-mediated inflammation associated with autoimmune disorders including, but not limited to, myasthenia gravis, Alzheimer's disease, multiple sclerosis, rheumatoid arthritis, systemic lupus erythematosus, insulin-dependent diabetes mellitus, acute disseminated encephalomyelitis, Addison's disease, antiphospholipid antibody syndrome, autoimmune hepatitis, Crohn's disease, Goodpasture's syndrome, Graves' disease, Guillain-Barre syndrome, Hashimoto's disease, idiopathic thrombocytopenic purpura, pemphigus, Sjogren's syndrome, and Takayasu's arteritis, may also be detected with the methods described herein.

Neurodegenerative condition (or disorder) is an inclusive term encompassing acute and chronic conditions, disorders or diseases of the central or peripheral nervous system. A neurodegenerative condition may be age-related, or it may result from injury or trauma, or it may be related to a specific disease or disorder. Acute neurodegenerative conditions include, but are not limited to, conditions associated with neuronal cell death or compromise including cerebrovascular insufficiency, e.g. due to stroke, focal or diffuse brain trauma, diffuse brain damage, spinal cord injury or peripheral nerve trauma, e.g., resulting from physical or chemical burns, deep cuts or limb severance. Examples of acute neurodegenerative disorders are: cerebral ischemia or infarction including embolic occlusion and thrombotic occlusion, reperfusion following acute ischemia, perinatal hypoxic-ischemic injury, cardiac arrest, as well as intracranial hemorrhage of any type (such as epidural, subdural, subarachnoid and intracerebral), and intracranial and intravertebral lesions (such as contusion, penetration, shear, compression and laceration), as well as whiplash and shaken infant syndrome. Chronic neurodegenerative conditions include, but are not limited to, Alzheimer's disease, Pick's disease, diffuse Lewy body disease, progressive supranuclear palsy (Steel-Richardson syndrome), multisystem degeneration (Shy-Drager syndrome), chronic epileptic conditions associated with neurodegeneration, motor neuron diseases including amyotrophic lateral sclerosis, degenerative ataxias, cortical basal degeneration, ALS-Parkinson's-Dementia complex of Guam, subacute sclerosing panencephalitis, Huntington's disease, Parkinson's disease, synucleinopathies (including multiple system atrophy), primary progressive aphasia, striatonigral degeneration, Machado-Joseph disease/spinocerebellar ataxia type 3 and olivopontocerebellar degenerations, Gilles De La Tourette's disease, bulbar and pseudobulbar palsy, spinal and spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, familial dysautonomia (Riley-Day syndrome), and prion diseases (including, but not limited to Creutzfeldt-Jakob, Gerstmann-Straussler-Scheinker disease, Kuru and fatal familial insomnia), demyelination diseases and disorders including multiple sclerosis and hereditary diseases such as leukodystrophies.

Mesenchymal stem cells (“MSC”) were originally derived from the embryonal mesoderm and subsequently have been isolated from adult bone marrow and other adult tissues. They can be differentiated to form muscle, bone, cartilage, fat, marrow stroma, and tendon. Mesoderm also differentiates into visceral mesoderm which can give rise to cardiac muscle, smooth muscle, or blood islands consisting of endothelium and hematopoietic progenitor cells. The differentiation potential of the mesenchymal stem cells that have been described thus far is limited to cells of mesenchymal origin, including the best characterized mesenchymal stem cell (See Pittenger, et al. Science (1999) 284: 143-147 and U.S. Pat. No. 5,827,740 (SH2.sup.+ SH4.sup.+ CD29.sup.+ CD44.sup.+ CD71.sup.+ CD90.sup.+ CD106.sup.+ CD120a.sup.+ CD124.sup.+ CD14.sup.− CD34.sup.− CD45.sup.−)). The invention teaches the use of various mesenchymal stem cells

In one embodiment MSC donor lots are generated from umbilical cord tissue. Means of generating umbilical cord tissue MSC have been previously published and are incorporated by reference [1-7]. The term “umbilical tissue derived cells (UTC)” refers, for example, to cells as described in U.S. Pat. No. 7,510,873, U.S. Pat. No. 7,413,734, U.S. Pat. No. 7,524,489, and U.S. Pat. No. 7,560,276. The UTC can be of any mammalian origin e.g. human, rat, primate, porcine and the like. In one embodiment of the invention, the UTC are derived from human umbilicus. umbilicus-derived cells, which relative to a human cell that is a fibroblast, a mesenchymal stem cell, or an iliac crest bone marrow cell, have reduced expression of genes for one or more of: short stature homeobox 2; heat shock 27 kDa protein 2; chemokine (C-X-C motif) ligand 12 (stromal cell-derived factor 1); elastin (supravalvular aortic stenosis, Williams-Beuren syndrome); Homo sapiens mRNA; cDNA DKFZp586M2022 (from clone DKFZp586M2022); mesenchyme homeobox 2 (growth arrest-specific homeobox); sine oculis homeobox homolog 1 (Drosophila); crystallin, alpha B; disheveled associated activator of morphogenesis 2; DKFZP586B2420 protein; similar to neuralin 1; tetranectin (plasminogen binding protein); src homology three (SH3) and cysteine rich domain; cholesterol 25-hydroxylase; runt-related transcription factor 3; interleukin 11 receptor, alpha; procollagen C-endopeptidase enhancer; frizzled homolog 7 (Drosophila); hypothetical gene BC008967; collagen, type VIII, alpha 1; tenascin C (hexabrachion); iroquois homeobox protein 5; hephaestin; integrin, beta 8; synaptic vesicle glycoprotein 2; neuroblastoma, suppression of tumorigenicity 1; insulin-like growth factor binding protein 2, 36 kDa; Homo sapiens cDNA FLJ12280 fis, clone MAMMA1001744; cytokine receptor-like factor 1; potassium intermediate/small conductance calcium-activated channel, subfamily N, member 4; integrin, beta 7; transcriptional co-activator with PDZ-binding motif (TAZ); sine oculis homeobox homolog 2 (Drosophila); KIAA1034 protein; vesicle-associated membrane protein 5 (myobrevin); EGF-containing fibulin-like extracellular matrix protein 1; early growth response 3; distal-less homeobox 5; hypothetical protein FLJ20373; aldo-keto reductase family 1, member C3 (3-alpha hydroxysteroid dehydrogenase, type II); biglycan; transcriptional co-activator with PDZ-binding motif (TAZ); fibronectin 1; proenkephalin; integrin, beta-like 1 (with EGF-like repeat domains); Homo sapiens mRNA full length insert cDNA clone EUROIMAGE 1968422; EphA3; KIAA0367 protein; natriuretic peptide receptor C/guanylate cyclase C (atrionatriuretic peptide receptor C); hypothetical protein FLJ14054; Homo sapiens mRNA; cDNA DKFZp564B222 (from clone DKFZp564B222); BCL2/adenovirus E1B 19 kDa interacting protein 3-like; AE binding protein 1; and cytochrome c oxidase subunit VIIa polypeptide 1 (muscle). In addition, these isolated human umbilicus-derived cells express a gene for each of interleukin 8; reticulon 1; chemokine (C-X-C motif) ligand 1 (melonoma growth stimulating activity, alpha); chemokine (C-X-C motif) ligand 6 (granulocyte chemotactic protein 2); chemokine (C-X-C motif) ligand 3; and tumor necrosis factor, alpha-induced protein 3, wherein the expression is increased relative to that of a human cell which is a fibroblast, a mesenchymal stem cell, an iliac crest bone marrow cell, or placenta-derived cell. The cells are capable of self-renewal and expansion in culture, and have the potential to differentiate into cells of other phenotypes.

Methods of deriving cord tissue mesenchymal stem cells from human umbilical tissue are provided. The cells are capable of self-renewal and expansion in culture, and have the potential to differentiate into cells of other phenotypes. The method comprises (a) obtaining human umbilical tissue; (b) removing substantially all of blood to yield a substantially blood-free umbilical tissue, (c) dissociating the tissue by mechanical or enzymatic treatment, or both, (d) resuspending the tissue in a culture medium, and (e) providing growth conditions which allow for the growth of a human umbilicus-derived cell capable of self-renewal and expansion in culture and having the potential to differentiate into cells of other phenotypes.

Tissue can be obtained from any completed pregnancy, term or less than term, whether delivered vaginally, or through other routes, for example surgical Cesarean section. Obtaining tissue from tissue banks is also considered within the scope of the present invention.

The tissue is rendered substantially free of blood by any means known in the art. For example, the blood can be physically removed by washing, rinsing, and diluting and the like, before or after bulk blood removal for example by suctioning or draining. Other means of obtaining a tissue substantially free of blood cells might include enzymatic or chemical treatment.

Dissociation of the umbilical tissues can be accomplished by any of the various techniques known in the art, including by mechanical disruption, for example, tissue can be aseptically cut with scissors, or a scalpel, or such tissue can be otherwise minced, blended, ground, or homogenized in any manner that is compatible with recovering intact or viable cells from human tissue.

In a presently preferred embodiment, the isolation procedure also utilizes an enzymatic digestion process. Many enzymes are known in the art to be useful for the isolation of individual cells from complex tissue matrices to facilitate growth in culture. As discussed above, a broad range of digestive enzymes for use in cell isolation from tissue is available to the skilled artisan. Ranging from weakly digestive (e.g. deoxyribonucleases and the neutral protease, dispase) to strongly digestive (e.g. papain and trypsin), such enzymes are available commercially. A nonexhaustive list of enzymes compatable herewith includes mucolytic enzyme activities, metalloproteases, neutral proteases, serine proteases (such as trypsin, chymotrypsin, or elastase), and deoxyribonucleases. Presently preferred are enzyme activities selected from metalloproteases, neutral proteases and mucolytic activities. For example, collagenases are known to be useful for isolating various cells from tissues. Deoxyribonucleases can digest single-stranded DNA and can minimize cell-clumping during isolation. Enzymes can be used alone or in combination. Serine protease are preferably used in a sequence following the use of other enzymes as they may degrade the other enzymes being used. The temperature and time of contact with serine proteases must be monitored. Serine proteases may be inhibited with alpha 2 microglobulin in serum and therefore the medium used for digestion is preferably serum-free. EDTA and DNase are commonly used and may improve yields or efficiencies. Preferred methods involve enzymatic treatment with for example collagenase and dispase, or collagenase, dispase, and hyaluronidase, and such methods are provided wherein in certain preferred embodiments, a mixture of collagenase and the neutral protease dispase are used in the dissociating step. More preferred are those methods which employ digestion in the presence of at least one collagenase from Clostridium histolyticum, and either of the protease activities, dispase and thermolysin. Still more preferred are methods employing digestion with both collagenase and dispase enzyme activities. Also preferred are methods which include digestion with a hyaluronidase activity in addition to collagenase and dispase activities. The skilled artisan will appreciate that many such enzyme treatments are known in the art for isolating cells from various tissue sources. For example, the LIBERASE BLENDZYME (Roche) series of enzyme combinations of collagenase and neutral protease are very useful and may be used in the instant methods. Other sources of enzymes are known, and the skilled artisan may also obtain such enzymes directly from their natural sources. The skilled artisan is also well-equipped to assess new, or additional enzymes or enzyme combinations for their utility in isolating the cells of the invention. Preferred enzyme treatments are 0.5, 1, 1.5, or 2 hours long or longer. In other preferred embodiments, the tissue is incubated at 37.degree. C. during the enzyme treatment of the dissociation step. Diluting the digest may also improve yields of cells as cells may be trapped within a viscous digest.

While the use of enzyme activites is presently preferred, it is not required for isolation methods as provided herein. Methods based on mechanical separation alone may be successful in isolating the instant cells from the umbilicus as discussed above.

The cells can be resuspended after the tissue is dissociated into any culture medium as discussed herein above. Cells may be resuspended following a centrifugation step to separate out the cells from tissue or other debris. Resuspension may involve mechanical methods of resuspending, or simply the addition of culture medium to the cells.

Providing the growth conditions allows for a wide range of options as to culture medium, supplements, atmospheric conditions, and relative humidity for the cells. A preferred temperature is 37.degree. C., however the temperature may range from about 35.degree. C. to 39.degree. C. depending on the other culture conditions and desired use of the cells or culture.

Presently preferred are methods which provide cells which require no exogenous growth factors, except as are available in the supplemental serum provided with the Growth Medium. Also provided herein are methods of deriving umbilical cells capable of expansion in the absence of particular growth factors. The methods are similar to the method above, however they require that the particular growth factors (for which the cells have no requirement) be absent in the culture medium in which the cells are ultimately resuspended and grown in. In this sense, the method is selective for those cells capable of division in the absence of the particular growth factors. Preferred cells in some embodiments are capable of growth and expansion in chemically-defined growth media with no serum added. In such cases, the cells may require certain growth factors, which can be added to the medium to support and sustain the cells. Presently preferred factors to be added for growth on serum-free media include one or more of FGF, EGF, IGF, and PDGF. In more preferred embodiments, two, three or all four of the factors are add to serum free or chemically defined media. In other embodiments, LIF is added to serum-free medium to support or improve growth of the cells.

Also provided are methods wherein the cells can expand in the presence of from about 5% to about 20% oxygen in their atmosphere. Methods to obtain cells that require L-valine require that cells be cultured in the presence of L-valine. After a cell is obtained, its need for L-valine can be tested and confirmed by growing on D-valine containing medium that lacks the L-isomer.

Methods are provided wherein the cells can undergo at least 25, 30, 35, or 40 doublings prior to reaching a senescent state. Methods for deriving cells capable of doubling to reach 10.sup.14 cells or more are provided. Preferred are those methods which derive cells that can double sufficiently to produce at least about 10.sup.14, 10.sup.15, 10.sup.16, or 10.sup.17 or more cells when seeded at from about 10.sup.3 to about 10.sup.6 cells/cm.sup.2 in culture. Preferably these cell numbers are produced within 80, 70, or 60 days or less. In one embodiment, cord tissue mesenchymal stem cells are isolated and expanded, and possess one or more markers selected from a group comprising of CD10, CD13, CD44, CD73, CD90, CD141, PDGFr-alpha, or HLA-A,B,C. In addition, the cells do not produce one or more of CD31, CD34, CD45, CD117, CD141, or HLA-DR,DP, DQ.

In one embodiment, bone marrow MSC lots are generated, means of generating BM MSC are known in the literature and examples are incorporated by reference.

In one embodiment BM-MSC are generated as follows

1. 500 mL Isolation Buffer is prepared (PBS+2% FBS+2 mM EDTA) using sterile components or filtering Isolation Buffer through a 0.2 micron filter. Once made, the Isolation Buffer was stored at 2-8.degree. C.

2. The total number of nucleated cells in the BM sample is counted by taking 10 .mu.L BM and diluting it 1/50-1/100 with 3% Acetic Acid with Methylene Blue (STEMCELL Catalog #07060). Cells are counted using a hemacytometer.

3. 50 mL Isolation Buffer is warmed to room temperature for 20 minutes prior to use and bone marrow was diluted 5/14 final dilution with room temperature Isolation Buffer (e.g. 25 mL BM was diluted with 45 mL Isolation Buffer for a total volume of 70 mL).

4. In three 50 mL conical tubes (BD Catalog #352070), 17 mL Ficoll-Paque™ PLUS (Catalog #07907/07957) is pipetted into each tube. About 23 mL of the diluted BM from step 3 was carefully layered on top of the Ficoll-Paque™ PLUS in each tube.

5. The tubes are centrifuged at room temperature (15-25.degree. C.) for 30 minutes at 300.times.g in a bench top centrifuge with the brake off.

6. The upper plasma layer is removed and discarded without disturbing the plasma:Ficoll-Paque™ PLUS interface. The mononuclear cells located at the interface layer are carefully removed and placed in a new 50 mL conical tube. Mononuclear cells are resuspended with 40 mL cold (2-8.degree. C.) Isolation Buffer and mixed gently by pipetting.

7. Cells were centrifuged at 300.times.g for 10 minutes at room temperature in a bench top centrifuge with the brake on. The supernatant is removed and the cell pellet resuspended in 1-2 mL cold Isolation Buffer.

8. Cells were diluted 1/50 in 3% Acetic Acid with Methylene Blue and the total number of nucleated cells counted using a hemacytometer.

9. Cells are diluted in Complete Human MesenCult®-Proliferation medium (STEMCELL catalog #05411) at a final concentration of 1.times.10.sup.6 cells/mL.

10. BM-derived cells were ready for expansion and CFU-F assays in the presence of GW2580, which can then be used for specific applications.

Said BM-MSC are treated with Noble gas containing mixtures at a concentration and frequency sufficient to enhance HIF-1 alpha activity, which is used as a marker of augmented activity.

In one embodiment, MSC are generated according to protocols previously utilized for treatment of patients utilizing bone marrow derived MSC. Specifically, bone marrow is aspirated (10-30 ml) under local anesthesia (with or without sedation) from the posterior iliac crest, collected into sodium heparin containing tubes and transferred to a Good Manufacturing Practices (GMP) clean room. Bone marrow cells are washed with a washing solution such as Dulbecco's phosphate-buffered saline (DPBS), RPMI, or PBS supplemented with autologous patient plasma and layered on to 25 ml of Percoll (1.073 g/ml) at a concentration of approximately 1-2′10⁷ cells/ml. Subsequently the cells are centrifuged at 900 g for approximately 30 min or a time period sufficient to achieve separation of mononuclear cells from debris and erythrocytes. Said cells are then washed with PBS and plated at a density of approximately 1′10⁶ cells per ml in 175 cm² tissue culture flasks in DMEM with 10% FCS with flasks subsequently being loaded with a minimum of 30 million bone marrow mononuclear cells. The MSCs are allowed to adhere for 72 h followed by media changes every 3-4 days. Adherent cells are removed with 0.05% trypsin-EDTA and replated at a density of 1′10⁶ per 175 cm². Said bone marrow MSC may be administered intravenously, or in a preferred embodiment, intrathecally in a patient suffering radiation associated neurodegenerative manifestations. Although doses may be determined by one of skill in the art, and are dependent on various patient characteristics, intravenous administration may be performed at concentrations ranging from 1-10 million MSC per kilogram, with a preferred dose of approximately 2-5 million cells per kilogram.

In some embodiments of the invention MSC are transferred to possess enhanced neuromodulatory and neuroprotective properties. Said transfection may be accomplished by use of lentiviral vectors, said means to perform lentiviral mediated transfection are well-known in the art and discussed in the following references [8-14]. Some specific examples of lentiviral based transfection of genes into MSC include transfection of SDF-1 to promote stem cell homing, particularly hematopoietic stem cells [15], GDNF to treat Parkinson's in an animal model [16], HGF to accelerate remyelination in a brain injury model [17], akt to protect against pathological cardiac remodeling and cardiomyocyte death [18], TRAIL to induce apoptosis of tumor cells [19-22], PGE-1 synthase for cardioprotection [23], NUR77 to enhance migration [24], BDNF to reduce ocular nerve damage in response to hypertension [25], HIF-1 alpha to stimulate osteogenesis [26], dominant negative CCL2 to reduce lung fibrosis [27], interferon beta to reduce tumor progression [28], HLA-G to enhance immune suppressive activity [29], hTERT to induce differentiation along the hepatocyte lineage [30], cytosine deaminase [31], OCT-4 to reduce senescence [32, 33], BAMBI to reduce TGF expression and protumor effects [34], HO-1 for radioprotection [35], LIGHT to induce antitumor activity [36], miR-126 to enhance angiogenesis [37, 38], bcl-2 to induce generation of nucleus pulposus cells [39], telomerase to induce neurogenesis [40], CXCR4 to accelerate hematopoietic recovery [41] and reduce unwanted immunity [42], wnt11 to promote regenerative cytokine production [43], and the HGF antagonist NK4 to reduce cancer [44].

Cell cultures are tested for sterility weekly, endotoxin by limulus amebocyte lysate test, and mycoplasma by DNA-fluorochrome stain.

In order to determine the quality of MSC cultures, flow cytometry is performed on all cultures for surface expression of SH-2, SH-3, SH-4 MSC markers and lack of contaminating CD14- and CD-45 positive cells. Cells were detached with 0.05% trypsin-EDTA, washed with DPBS+2% bovine albumin, fixed in 1% paraformaldehyde, blocked in 10% serum, incubated separately with primary SH-2, SH-3 and SH-4 antibodies followed by PE-conjugated anti-mouse IgG(H+L) antibody. Confluent MSC in 175 cm² flasks are washed with Tyrode's salt solution, incubated with medium 199 (M199) for 60 min, and detached with 0.05% trypsin-EDTA (Gibco). Cells from 10 flasks were detached at a time and MSCs were resuspended in 40 ml of M199 +1% human serum albumin (HSA; American Red Cross, Washington DC, USA). MSCs harvested from each 10-flask set were stored for up to 4 h at 4° C. and combined at the end of the harvest. A total of 2-10, 10⁶ MSC/kg were resuspended in M199 +1% HSA and centrifuged at 460 g for 10 min at 20° C. Cell pellets were resuspended in fresh M199+1% HSA media and centrifuged at 460 g for 10 min at 20° C. for three additional times. Total harvest time was 2-4 h based on MSC yield per flask and the target dose. Harvested MSC were cryopreserved in Cryocyte (Baxter, Deerfield, Ill., USA) freezing bags using a rate controlled freezer at a final concentration of 10% DMSO (Research Industries, Salt Lake City, Utah, USA) and 5% HSA. On the day of infusion cryopreserved units were thawed at the bedside in a 37° C. water bath and transferred into 60 ml syringes within 5 min and infused intravenously into patients over 10-15 min. Patients are premedicated with 325-650 mg acetaminophen and 12.5-25 mg of diphenhydramine orally. Blood pressure, pulse, respiratory rate, temperature and oxygen saturation are monitored at the time of infusion and every 15 min thereafter for 3 h followed by every 2 h for 6 h.

In one embodiment of the invention enhanced MSC are transfected with anti-apoptotic proteins to enhance in vivo longevity. The present invention includes a method of using MSC that have been cultured under conditions to express increased amounts of at least one anti-apoptotic protein as a therapy to inhibit or prevent apoptosis. In one embodiment, the MSC which are used as a therapy to inhibit or prevent apoptosis have been contacted with an apoptotic cell. The invention is based on the discovery that MSC that have been contacted with an apoptotic cell express high levels of anti-apoptotic molecules. In some instances, the MSC that have been contacted with an apoptotic cell secrete high levels of at least one anti-apoptotic protein, including but not limited to, STC-1, BCL-2, XIAP, Survivin, and Bcl-2XL. Methods of transfecting antiapoptotic genes into MSC have been previously described which can be applied to the current invention, said antiapoptotic genes that can be utilized for practice of the invention, in a nonlimiting way, include GATA-4 [45], FGF-2 [46], bcl-2 [39, 47], and HO-1 [48]. Based upon the disclosure provided herein, MSC can be obtained from any source. The MSC may be autologous with respect to the recipient (obtained from the same host) or allogeneic with respect to the recipient. In addition, the MSC may be xenogeneic to the recipient (obtained from an animal of a different species). In one embodiment of the invention MSC are pretreated with agents to induce expression of antiapoptotic genes, one example is pretreatment with exendin-4 as previously described [49]. In a further non-limiting embodiment, MSC used in the present invention can be isolated, from the bone marrow of any species of mammal, including but not limited to, human, mouse, rat, ape, gibbon, bovine. In a non-limiting embodiment, the MSC are isolated from a human, a mouse, or a rat. In another non-limiting embodiment, the MSC are isolated from a human.

Based upon the present disclosure, MSC can be isolated and expanded in culture in vitro to obtain sufficient numbers of cells for use in the methods described herein provided that the MSC are cultured in a manner that promotes contact with a tumor endothelial cell. For example, MSC can be isolated from human bone marrow and cultured in complete medium (DMEM low glucose containing 4 mM L-glutamine, 10% FBS, and 1% penicillin/streptomycin) in hanging drops or on non-adherent dishes. The invention, however, should in no way be construed to be limited to any one method of isolating and/or to any culturing medium. Rather, any method of isolating and any culturing medium should be construed to be included in the present invention provided that the MSC are cultured in a manner that provides MSC to express increased amounts of at least one anti-apoptotic protein. Culture conditions for growth of clinical grade MSC have been described in the literature and are incorporated by reference [50-83].

Without being limited to any one or more explanatory mechanisms for the immunomodulatory, regenerative and other properties, activities, and effects of enhanced MSC, it is worth nothing that they can modulate immune responses through a variety of modalities. For instance, enhanced MSC can have direct effects on a graft or host. Such direct effects are primarily a matter of direct contact between enhanced MSC and cells of the host or graft. The contact may be with structural members of the cells or with constituents in their immediate environment. Such direct mechanisms may involve direct contact, diffusion, uptake, or other processes well known to those skilled in the art. The direct activities and effects of the enhanced MSC may be limited spatially, such as to an area of local deposition or to a bodily compartment accessed by injection.

Enhanced MSC also can “home” in response to “homing” signals, such as those released at sites of injury or disease. Since homing often is mediated by signals whose natural function is to recruit cells to the sites where repairs are needed, the homing behavior can be a powerful tool for concentrating Enhanced MSC to therapeutic targets. This effect can be stimulated by specific factors, as discussed below.

Enhanced MSC may also modulate immune processes by their response to factors. This may occur additionally or alternatively to direct modulation. Such factors may include homing factors, mitogens, and other stimulatory factors. They may also include differentiation factors, and factors that trigger particular cellular processes. Among the latter are factors that cause the secretion by cells of other specific factors, such as those that are involved in recruiting cells, such as stem cells (including Enhanced MSC), to a site of injury or disease.

Enhanced MSC may, in addition to the foregoing or alternatively thereto, secrete factors that act on endogenous cells, such as stem cells or progenitor cells. The factors may act on other cells to engender, enhance, decrease, or suppress their activities. enhanced MSC may secrete factors that act on stem, progenitor, or differentiated cells causing those cells to divide and/or differentiate. One such factor is exosomes and microvesicles produced by said enhanced MSC. Enhanced MSC that home to a site where repair is needed may secrete trophic factors that attract other cells to the site. In this way, Enhanced MSC may attract stem, progenitor, or differentiated cells to a site where they are needed. Enhanced MSC also may secrete factors that cause such cells to divide or differentiate. Secretion of such factors, including trophic factors, can contribute to the efficacy of enhanced MSC in, for instance, limiting inflammatory damage, limiting vascular permeability, improving cell survival, and engendering and/or augmenting homing of repair cells to sites of damage. Such factors also may affect T-cell proliferation directly. Such factors also may affect dendritic cells, by decreasing their phagocytic and antigen presenting activities, which also may affect T-cell activity. Furthermore such factors, or Enhanced MSC themselves, may be capable of modulating T regulatory cell numbers.

By these and other mechanisms, enhanced MSC can provide beneficial immunomodulatory effects, including, but not limited to, suppression of undesirable and/or deleterious immune reactions, responses, functions, diseases, and the like. Enhanced MSC in various embodiments of the invention provide beneficial immunomodulatory properties and effects that are useful by themselves or in adjunctive therapy for precluding, preventing, lessening, decreasing, ameliorating, mitigating, treating, eliminating and/or curing deleterious immune processes and/or conditions. Such processes and conditions include, for instance, autoimmune diseases, anemias, neoplasms, HVG, GVHD, and certain inflammatory disorders. In one particular embodiment, said enhanced MSC are useful for treatment of Neurological disease, inflammatory conditions, psychiatric disorders, inborn errors of metabolisms, vascular disease, cardiac disease, renal disease, hepatic disease, pulmonary disease, ocular conditions such as uveitis, gastrointestinal disorders, orthopedic disorders, dermal disorders, neoplasias, prevention of neoplasias, hematopoietic disorders, reproductive disorders, gynecological disorders, urological disorders, immunological disorders, olfactory disorders, and auricular disorders.

Enhanced MSC are useful in these other regards particularly in mammals. In various embodiments of the invention in this regard, Enhanced MSC are used therapeutically in human patients, often adjunctively to other therapies.

Enhanced MSC can be prepared from a variety of tissues, such as bone marrow cells, umbilical cord tissue, peripheral blood, mobilized peripheral blood, adipose tissue, menstrual blood and other tissue sources known to contain MSC. When tissue sources of MSC are used said tissue isolates from which the Enhanced MSC are isolated comprise a mixed populations of cells. Enhanced MSC constitute a very small percentage in these initial populations. They must be purified away from the other cells before they can be expanded in culture sufficiently to obtain enough cells for therapeutic applications.

In some embodiments the enhanced MSC preparations are clonally derived. In principle, the Enhanced MSC in these preparations are genetically identical to one another and, if properly prepared and maintained, are free of other cells. In some embodiments enhanced MSC preparations that are less pure than these may be used. While rare, less pure populations may arise when the initial cloning step requires more than one cell. If these are not all enhanced MSC, expansion will produce a mixed population in which enhanced MSC are only one of at least two types of cells. More often mixed populations arise when enhanced MSC are administered in admixture with one or more other types of cells.

In many embodiments the purity of enhanced MSC for administration to a subject is about 100%. In other embodiments it is 95% to 100%. In some embodiments it is 85% to 95%. Particularly in the case of admixtures with other cells, the percentage of Enhanced MSC can be 25%-30%, 30%-35%, 35%-40%, 40%-45%, 45%-50%, 60%-70%, 70%-80%, 80%-90%, or 90%-95%.

The number of enhanced MSC in a given volume can be determined by well known and routine procedures and instrumentation. The percentage of enhanced MSC in a given volume of a mixture of cells can be determined by much the same procedures. Cells can be readily counted manually or by using an automatic cell counter. Specific cells can be determined in a given volume using specific staining and visual examination and by automated methods using specific binding reagent, typically antibodies, fluorescent tags, and a fluorescence activated cell sorter.

Enhanced MSC immunomodulation may involve undifferentiated enhanced MSC. It may involve enhanced MSC that are committed to a differentiation pathway. Such immunomodulation also may involve enhanced MSC that have differentiated into a less potent stem cell with limited differentiation potential. It also may involve enhanced MSC that have differentiated into a terminally differentiated cell type. The best type or mixture of enhanced MSC will be determined by the particular circumstances of their use, and it will be a matter of routine design for those skilled in the art to determine an effective type or combination of enhanced MSC.

Various aspects of the invention of the invention relating to the above are enumerated in the following paragraphs:

Aspect 1. A method of generating mesenchymal stem cells with enhanced therapeutic activity, said method comprising treatment of mesenchymal stem cells with one or more Noble gases at a concentration and frequency sufficient to induce more than 5% increase in HIF-1 alpha expression in said mesenchymal stem cell.

Aspect 2. The method of aspect 1, wherein said mesenchymal stem cells are naturally occurring mesenchymal stem cells.

Aspect 3. The method of aspect 1, wherein said mesenchymal stem cells are generated in vitro.

Aspect 4. The method of aspect 2, wherein said naturally occurring mesenchymal stem cells are tissue derived.

Aspect 5. The method of aspect 2, wherein said naturally occurring mesenchymal stem cells are derived from a bodily fluid.

Aspect 6. The method of aspect 4, wherein said tissue derived mesenchymal stem cells are selected from a group comprising of: a) bone marrow; b) perivascular tissue; c) adipose tissue; d) placental tissue; e) amniotic membrane; f) omentum; g) tooth; h) umbilical cord tissue; i) fallopian tube tissue; j) hepatic tissue; k) renal tissue; l) cardiac tissue; m) tonsillar tissue; n) testicular tissue; o) ovarian tissue; p) neuronal tissue; q) auricular tissue; r) colonic tissue; s) submucosal tissue; t) hair follicle tissue; u) pancreatic tissue; v) skeletal muscle tissue; and w) subepithelial umbilical cord tissue.

Aspect 7. The method of aspect 4, wherein said tissue derived mesenchymal stem cells are isolated from tissues containing cells selected from a group of cells comprising of: endothelial cells, epithelial cells, dermal cells, endodermal cells, mesodermal cells, fibroblasts, osteocytes, chondrocytes, natural killer cells, dendritic cells, hepatic cells, pancreatic cells, stromal cells, salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells. bowman's gland cells, Brunner's gland cells, seminal vesicle cells, prostate gland cells, bulbourethral gland cells, Bartholin's gland cells, gland of Littre cells, uterus endometrium cells, isolated goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cell, oxyphil cell, adrenal gland cells, chromaffin cells, Leydig cells, theca interna cells, corpus luteum cells, granulosa lutein cells, theca lutein cells, juxtaglomerular cell, macula densa cells, peripolar cells, mesangial cell, blood vessel and lymphatic vascular endothelial fenestrated cells, blood vessel and lymphatic vascular endothelial continuous cells, blood vessel and lymphatic vascular endothelial splenic cells, synovial cells, serosal cell (lining peritoneal, pleural, and pericardial cavities), squamous cells, columnar cells, dark cells, vestibular membrane cell (lining endolymphatic space of ear), stria vascularis basal cells, stria vascularis marginal cell (lining endolymphatic space of ear), cells of Claudius, cells of Boettcher, choroid plexus cells, pia-arachnoid squamous cells, pigmented ciliary epithelium cells, nonpigmented ciliary epithelium cells, corneal endothelial cells, peg cells, respiratory tract ciliated cells, oviduct ciliated cell, uterine endometrial ciliated cells, rete testis ciliated cells, ductulus efferens ciliated cells, ciliated ependymal cells, epidermal keratinocytes, epidermal basal cells, keratinocyte of fingernails and toenails, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, surface epithelial cells of stratified squamous epithelium, basal cell of epithelia, urinary epithelium cells, auditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cell (blood pH sensor), type I hair cell of vestibular apparatus of ear (acceleration and gravity), type II hair cells of vestibular apparatus of ear, type I taste bud cells cholinergic neural cells, adrenergic neural cells, peptidergic neural cells, inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells, duct cells, intestinal brush border cells, exocrine gland striated duct cells, gall bladder epithelial cells, ductulus efferens nonciliated cells, epididymal principal cells, epididymal basal cells, ameloblast epithelial cells, planum semilunatum epithelial cells, organ of Corti interdental epithelial cells, loose connective tissue fibroblasts, corneal keratocytes, tendon fibroblasts, bone marrow reticular tissue fibroblasts, nonepithelial fibroblasts, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, stellate cells (ear), hepatic stellate cells (Ito cells), pancreatic stelle cells, red skeletal muscle cells, white skeletal muscle cells, intermediate skeletal muscle cells, nuclear bag cells of muscle spindle, nuclear chain cells of muscle spindle, satellite cells, ordinary heart muscle cells, nodal heart muscle cells, Purkinje fiber cells, smooth muscle cells, myoepithelial cells of iris, myoepithelial cell of exocrine glands, melanocytes, retinal pigmented epithelial cells, oogonia/oocytes, spermatids, spermatocytes, spermatogonium cells, spermatozoa, ovarian follicle cells, Sertoli cells, thymus epithelial cell, and/or interstitial kidney cells.

Aspect 8. The method of aspect 1, wherein said mesenchymal stem cells are plastic adherent.

Aspect 9. The method of aspect 1, wherein said mesenchymal stem cells express a marker selected from a group comprising of: a) CD73; b) CD90; and c) CD105.

Aspect 10. The method of aspect 1, wherein said mesenchymal stem cells lack expression of a marker selected from a group comprising of: a) CD14; b) CD45; and c) CD34.

Aspect 11. The method of aspect 6, wherein said mesenchymal stem cells from umbilical cord tissue express markers selected from a group comprising of; a) oxidized low density lipoprotein receptor 1, b) chemokine receptor ligand 3; and c) granulocyte chemotactic protein.

Aspect 12. The method of aspect 6, wherein said mesenchymal stem cells from umbilical cord tissue do not express markers selected from a group comprising of: a) CD117; b) CD31; c) CD34; and CD45;

Aspect 13. The method of aspect 6, wherein said mesenchymal stem cells from umbilical cord tissue express, relative to a human fibroblast, increased levels of interleukin 8 and reticulon 1

Aspect 14. The method of aspect 6, wherein said mesenchymal stem cells from umbilical cord tissue have the potential to differentiate into cells of at least a skeletal muscle, vascular smooth muscle, pericyte or vascular endothelium phenotype.

Aspect 15. The method of aspect 6, wherein said mesenchymal stem cells from umbilical cord tissue express markers selected from a group comprising of: a) CD10; b) CD13; c) CD44; d) CD73; and e) CD90.

Aspect 16. The method of aspect 6, wherein said umbilical cord tissue mesenchymal stem cell is an isolated umbilical cord tissue cell isolated from umbilical cord tissue substantially free of blood that is capable of self-renewal and expansion in culture,

Aspect 17. The method of aspect 16, wherein said umbilical cord tissue mesenchymal stem cells has the potential to differentiate into cells of other phenotypes.

Aspect 18. The method of aspect 17, wherein said other phenotypes comprise: a) osteocytic; b) adipogenic; and c) chondrogenic differentiation.

Aspect 19. The method of aspect 6, wherein said cord tissue derived mesenchymal stem cells can undergo at least 20 doublings in culture.

Aspect 20. The method of aspect 6, wherein said cord tissue derived mesenchymal stem cell maintains a normal karyotype upon passaging

Aspect 21. The method of aspect 6, wherein said cord tissue derived mesenchymal stem cell expresses a marker selected from a group of markers comprised of: a) CD10 b) CD13; c) CD44; d) CD73; e) CD90; f) PDGFr-alpha; g) PD-L2; and h) HLA-A,B,C

Aspect 22. The method of aspect 6, wherein said cord tissue mesenchymal stem cells does not express one or more markers selected from a group comprising of; a) CD31; b) CD34; c) CD45; d) CD80; e) CD86; f) CD117; g) CD141; h) CD178; i) B7-H2; j) HLA-G and k) HLA-DR,DP,DQ.

Aspect 23. The method of aspect 6, wherein said umbilical cord tissue-derived cell secretes factors selected from a group comprising of: a) MCP-1; b) MIP1beta; c) IL-6; d) IL-8; e) GCP-2; f) HGF; g) KGF; h) FGF; i) HB-EGF; j) BDNF; k) TPO; 1) RANTES; and m) TIMP1

Aspect 24. The method of aspect 6, wherein said umbilical cord tissue derived cells express markers selected from a group comprising of: a) TRA1-60; b) TRA1-81; c) SSEA3; d) SSEA4; and e) NANOG.

Aspect 25. The method of aspect 6, wherein said umbilical cord tissue-derived cells are positive for alkaline phosphatase staining.

Aspect 26. The method of aspect 6, wherein said umbilical cord tissue-derived cells are capable of differentiating into one or more lineages selected from a group comprising of; a) ectoderm; b) mesoderm, and; c) endoderm.

Aspect 27. The method of aspect 6, wherein said bone marrow derived mesenchymal stem cells possess markers selected from a group comprising of: a) CD73; b) CD90; and c) CD105.

Aspect 28. The method of aspect 6, wherein said bone marrow derived mesenchymal stem cells possess markers selected from a group comprising of: a) LFA-3; b) ICAM-1; c) PECAM-1; d) P-selectin; e) L-selectin; f) CD49b/CD29; g) CD49c/CD29; h) CD49d/CD29; i) CD29; j) CD18; k) CD61; 1) 6-19; m) thrombomodulin; n) telomerase; o) CD10; p) CD13; and q) integrin beta.

Aspect 29. The method of aspect 6, wherein said bone marrow derived mesenchymal stem cell is a mesenchymal stem cell progenitor cell.

Aspect 30. The method of aspect 29, wherein said mesenchymal progenitor cells are a population of bone marrow mesenchymal stem cells enriched for cells containing STRO-1

Aspect 31. The method of aspect 30, wherein said mesenchymal progenitor cells express both STRO-1 and VCAM-1.

Aspect 32. A method of aspect 30, wherein said STRO-1 expressing cells are negative for at least one marker selected from the group consisting of: a) CBFA-1; b) collagen type II; c) PPAR.gamma2; d) osteopontin; e) osteocalcin; f) parathyroid hormone receptor; g) leptin; h) H-ALBP; i) aggrecan; j) Ki67, and k) glycophorin A.

Aspect 33. The method of aspect 6, wherein said bone marrow mesenchymal stem cells lack expression of CD14, CD34, and CD45.

Aspect 34. The method of aspect 32, wherein said STRO-1 expressing cells are positive for a marker selected from a group comprising of: a) VACM-1; b) TKY-1; c) CD146 and; d) STRO-2

Aspect 35. The method of aspect 6, wherein said bone marrow mesenchymal stem cell express markers selected from a group comprising of: a) CD13; b) CD34; c) CD56 and; d) CD117

Aspect 36. The method of aspect 35, wherein said bone marrow mesenchymal stem cells do not express CD10.

Aspect 37. The method of aspect 35, wherein said bone marrow mesenchymal stem cells do not express CD2, CD5, CD14, CD19, CD33, CD45, and DRII.

Aspect 38. The method of aspect 35, wherein said bone marrow mesenchymal stem cells express CD13,CD34, CD56, CD90, CD117 and nestin, and which do not express CD2, CD3, CD10, CD14, CD16, CD31, CD33, CD45 and CD64.

Aspect 39. The method of aspect 6, wherein said skeletal muscle stem cells express markers selected from a group comprising of: a) CD13; b) CD34; c) CD56 and; d) CD117

Aspect 40. The method of aspect 40, wherein said skeletal muscle mesenchymal stem cells do not express CD10.

Aspect 41. The method of aspect 40, wherein said skeletal muscle mesenchymal stem cells do not express CD2, CD5, CD14, CD19, CD33, CD45, and DRII.

Aspect 42. The method of aspect 40, wherein said bone marrow mesenchymal stem cells express CD13,CD34, CD56, CD90, CD117 and nestin, and which do not express CD2, CD3, CD10, CD14, CD16, CD31, CD33, CD45 and CD64.

Aspect 43. The method of aspect 6, wherein said subepithelial umbilical cord derived mesenchymal stem cells possess markers selected from a group comprising of; a) CD29; b) CD73; c) CD90; d) CD166; e) SSEA4; f) CD9; g) CD44; h) CD146; and i) CD105

Aspect 44. The method of aspect 43, wherein said subepithelial umbilical cord derived mesenchymal stem cells do not express markers selected from a group comprising of; a)CD45; b) CD34; c) CD14; d) CD79; e) CD106; f) CD86; g) CD80; h) CD19; i) CD117; j) Stro-1 and k) HLA-DR.

Aspect 45. The method of aspect 43, wherein said subepithelial umbilical cord derived mesenchymal stem cells express CD29, CD73, CD90, CD166, SSEA4, CD9, CD44, CD146, and CD105.

Aspect 46. The method of aspect 43, wherein said subepithelial umbilical cord derived mesenchymal stem cells do not express CD45, CD34, CD14, CD79, CD106, CD86, CD80, CD19, CD117, Stro-1, and HLA-DR.

Aspect 47. The method of aspect 43, wherein said subepithelial umbilical cord derived mesenchymal stem cells are positive for SOX2.

Aspect 48. The method of aspect 43, wherein said subepithelial umbilical cord derived mesenchymal stem cells are positive for OCT4.

Aspect 49. The method of aspect 43, wherein said subepithelial umbilical cord derived mesenchymal stem cells are positive for OCT4 and SOX2.

Aspect 50. The method of aspect 1, wherein said efficacy reflects enhanced angiogenic activity.

Aspect 51. The method of aspect 1, wherein said efficacy reflects enhanced regenerative activity.

Aspect 52. The method of aspect 1, wherein said efficacy reflects enhanced ability to stimulate endogenous regenerative activity.

Aspect 53. The method of aspect 1, wherein said efficacy reflects enhanced ability to induce immune modulation.

Aspect 54. The method of aspect 1, wherein said efficacy reflects enhanced ability to induce clinical response in a disease condition.

Aspect 55. The method of aspect 54, wherein said disease condition is selected from a group comprising of: a) neurological disease; b) inflammatory conditions; c) psychiatric disorders; d) inborn errors of metabolisms; e) vascular disease; f) cardiac disease; g) renal disease; h) hepatic disease; i) pulmonary disease; j) ocular conditions; k) gastrointestinal disorders; l) orthopedic disorders; m) dermal disorders; n) neoplasia; o) predisposition to neoplasia; p) hematopoietic disorders; q) reproductive disorders; r) gynecological disorders; s) urological disorders; t) immunological disorders; u) olfactory disorders; and v) auricular disorders.

Aspect 56. The method of aspect 1, wherein said mesenchymal stem cells selected for enhanced efficacy are utilized as a source of conditioned media.

Aspect 57. The method of aspect 56, wherein conditioned media is used therapeutically in the treatment of a disorder.

Aspect 58. The method of aspect 57, wherein said disorder is selected from a group comprising of a) neurological disease; b) inflammatory conditions; c) psychiatric disorders; d) inborn errors of metabolisms; e) vascular disease; f) cardiac disease; g) renal disease; h) hepatic disease; i) pulmonary disease; j) ocular conditions; k) gastrointestinal disorders; l) orthopedic disorders; m) dermal disorders; n) neoplasia; o) predisposition to neoplasia; p) hematopoietic disorders; q) reproductive disorders; r) gynecological disorders; s) urological disorders; t) immunological disorders; u) olfactory disorders; and v) auricular disorders.

Aspect 59. The method of aspect 1, wherein said Noble gas is xenon.

Aspect 60. The method of aspect 59, wherein said Noble gas is a mixture, said mixture containing one or more of the following gases: a) nitrogen; b) helium; c) Nitric Oxide; d) krypton; e) argon; and f) neon.

Aspect 61. The method of aspect 1, wherein said mesenchymal stem cells are treated with said Noble gas containing mixture by incubating said cells in an atmosphere containing said gases.

Aspect 62. The method of aspect 61, wherein said atmosphere is created by means of a sealed enclosure.

Aspect 63. The method of aspect 62, wherein said sealed enclosure is an incubator.

Aspect 64. The method of aspect 1, wherein said Noble gas mixture containing said Noble gas is administered into culture media by dissolving of said Noble gas or mixture containing said Noble gas into said culture media.

Aspect 65. The method of aspect 1, wherein said noble gas containing mixture is comprised of a gas mixture containing oxygen and a proportion by volume of 20 to 70% of xenon.

Aspect 66. The method of aspect 65, wherein said proportion of xenon is between 22 and 60% by volume to oxygen.

Aspect 67. The method of aspect 65, wherein said proportion of xenon is between 25 and 60% by volume to oxygen.

Aspect 68. The method of aspect 1, wherein said noble gas containing mixture consists only of a) oxygen and xenon or b) air and xenon.

Aspect 69. The method of aspect 1, wherein said noble gas containing mixture also contains nitrogen, helium, Nitric Oxide, krypton, argon or neon.

Aspect 70. The method of aspect 1, wherein said noble gas containing mixture contains a proportion by volume of oxygen of between 15 and 25%.

Aspect 71. The method of aspect 1, wherein said noble gas containing mixture is supplied for inhalation from a pressurized container at a pressure greater than 2 bar.

Aspect 72. The method of aspect 1, wherein said noble gas containing mixture is administered intranasally.

Aspect 73. The method of aspect 1, wherein said noble gas containing mixture is administered through the use of a hyperbaric chamber.

Aspect 74. The method of aspect 73, wherein said hyperbaric chamber is pressurized to a pressure of no more than 3 atm (0.3 MPa).

Aspect 75. The method of aspect 73, wherein a noble gas is administered to the patient while the patient is in the hyperbaric environment.

Aspect 76. The method of aspect 1 wherein said noble gas is administered by inhalation or simulated inhalation.

Aspect 77. The method of aspect 1, wherein said noble gas is xenon, helium, or a mixture of xenon and helium.

Aspect 78. The method of aspect 1, wherein the noble gas is xenon or a mixture of xenon and helium, and the partial pressure of xenon is no more than about 0.8 atm (0.08 MPa).

Aspect 79. The method of aspect 1, wherein said noble gas is administered mixed with air, the air partial pressure being about 1 atm (0.1 MPa).

Aspect 80. The method of aspect 1, wherein said noble gas is administered as part of a gas mixture comprising oxygen, the nitrogen partial pressure in the mixture being equal to or less than about 0.8 atm (0.08 MPa).

Aspect 81. The method of aspect 80, wherein said gas mixture is essentially free of nitrogen.

Aspect 82. The method of aspect 80, wherein the oxygen partial pressure is about 0.2 atm (0.02 MPa).

### REFERENCES

1. Van Pham, P., et al., Isolation and proliferation of umbilical cord tissue derived mesenchymal stem cells for clinical applications. Cell Tissue Bank, 2015.

2. Fazzina, R., et al., A new standardized clinical-grade protocol for banking human umbilical cord tissue cells. Transfusion, 2015. 55(12): p. 2864-73.

3. Bieback, K., Platelet lysate as replacement for fetal bovine serum in mesenchymal stromal cell cultures. Transfus Med Hemother, 2013. 40(5): p. 326-35.

4. Stanko, P., et al., Comparison of human mesenchymal stem cells derived from dental pulp, bone marrow, adipose tissue, and umbilical cord tissue by gene expression. Biomed Pap Med Fac Univ Palacky Olomouc Czech Repub, 2014. 158(3): p. 373-7.

5. Schira, J., et al., Significant clinical, neuropathological and behavioural recovery from acute spinal cord trauma by transplantation of a well-defined somatic stem cell from human umbilical cord blood. Brain, 2012. 135(Pt 2): p. 431-46.

6. Hartmann, I., et al., Umbilical cord tissue-derived mesenchymal stem cells grow best under GMP-compliant culture conditions and maintain their phenotypic and functional properties. J Immunol Methods, 2010. 363(1): p. 80-9.

7. Friedman, R., et al., Umbilical cord mesenchymal stem cells: adjuvants for human cell transplantation. Biol Blood Marrow Transplant, 2007. 13(12): p. 1477-86.

8. Zhang, X. Y., et al., Lentiviral vectors for sustained transgene expression in human bone marrow-derived stromal cells. Mol Ther, 2002. 5(5 Pt 1): p. 555-65.

9. Kyriakou, C. A., et al., Human mesenchymal stem cells (hMSCs) expressing truncated soluble vascular endothelial growth factor receptor (tsFlk-1) following lentiviral-mediated gene transfer inhibit growth of Burkitt's lymphoma in a murine model. J Gene Med, 2006. 8(3): p. 253-64.

10. Worsham, D. N., et al., In vivo gene transfer into adult stem cells in unconditioned mice by in situ delivery of a lentiviral vector. Mol Ther, 2006. 14(4): p. 514-24.

11. Rabin, N., et al., A new xenograft model of myeloma bone disease demonstrating the efficacy of human mesenchymal stem cells expressing osteoprotegerin by lentiviral gene transfer. Leukemia, 2007. 21(10): p. 2181-91.

12. Kallifatidis, G., et al., Improved lentiviral transduction of human mesenchymal stem cells for therapeutic intervention in pancreatic cancer. Cancer Gene Ther, 2008. 15(4): p. 231-40.

13. Meyerrose, T. E., et al., Lentiviral-transduced human mesenchymal stem cells persistently express therapeutic levels of enzyme in a xenotransplantation model of human disease. Stem Cells, 2008. 26(7): p. 1713-22.

14. McGinley, L., et al., Lentiviral vector mediated modification of mesenchymal stem cells & enhanced survival in an in vitro model of ischaemia. Stem Cell Res Ther, 2011. 2(2): p. 12.

15. Liang, X., et al., Human bone marrow mesenchymal stem cells expressing SDF-1 promote hematopoietic stem cell function of human mobilised peripheral blood CD34+cells in vivo and in vitro. Int J Radiat Biol, 2010. 86(3): p. 230-7.

16. Glavaski-Joksimovic, A., et al., Glial cell line-derived neurotrophic factor-secreting genetically modified human bone marrow-derived mesenchymal stem cells promote recovery in a rat model of Parkinson's disease. J Neurosci Res, 2010. 88(12): p. 2669-81.

17. Liu, A. M., et al., Umbilical cord-derived mesenchymal stem cells with forced expression of hepatocyte growth factor enhance remyelination and functional recovery in a rat intracerebral hemorrhage model. Neurosurgery, 2010. 67(2): p. 357-65; discussion 365-6.

18. Yu, Y. S., et al., AKT-modified autologous intracoronary mesenchymal stem cells prevent remodeling and repair in swine infarcted myocardium. Chin Med J (Engl), 2010. 123(13): p. 1702-8.

19. Mueller, L. P., et al., TRAIL-transduced multipotent mesenchymal stromal cells (TRAIL-MSC) overcome TRAIL resistance in selected CRC cell lines in vitro and in vivo. Cancer Gene Ther, 2011. 18(4): p. 229-39.

20. Yan, C., et al., Suppression of orthotopically implanted hepatocarcinoma in mice by umbilical cord-derived mesenchymal stem cells with sTRAIL gene expression driven by AFP promoter. Biomaterials, 2014. 35(9): p. 3035-43.

21. Deng, Q., et al., TRAIL-secreting mesenchymal stem cells promote apoptosis in heat-shock-treated liver cancer cells and inhibit tumor growth in nude mice. Gene Ther, 2014. 21(3): p. 317-27.

22. Sage, E. K., et al., Systemic but not topical TRAIL-expressing mesenchymal stem cells reduce tumour growth in malignant mesothelioma. Thorax, 2014. 69(7): p. 638-47.

23. Lian, W. S., et al., In vivo therapy of myocardial infarction with mesenchymal stem cells modified with prostaglandin I synthase gene improves cardiac performance in mice. Life Sci, 2011. 88(9-10): p. 455-64.

24. Maijenburg, M. W., et al., Nuclear receptors Nur77 and Nurr1 modulate mesenchymal stromal cell migration. Stem Cells Dev, 2012. 21(2): p. 228-38.

25. Harper, M. M., et al., Transplantation of BDNF-secreting mesenchymal stem cells provides neuroprotection in chronically hypertensive rat eyes. Invest Ophthalmol Vis Sci, 2011. 52(7): p. 4506-15.

26. Zou, D., et al., In vitro study of enhanced osteogenesis induced by HIF-1 alpha-transduced bone marrow stem cells. Cell Prolif, 2011. 44(3): p. 234-43.

27. Saito, S., et al., Mesenchymal stem cells stably transduced with a dominant-negative inhibitor of CCL2 greatly attenuate bleomycin-induced lung damage. Am J Pathol, 2011. 179(3): p. 1088-94.

28. Seo, K. W., et al., Anti-tumor effects of canine adipose tissue-derived mesenchymal stromal cell-based interferon-beta gene therapy and cisplatin in a mouse melanoma model. Cytotherapy, 2011. 13(8): p. 944-55.

29. Yang, H. M., et al., Enhancement of the immunosuppressive effect of human adipose tissue-derived mesenchymal stromal cells through HLA-G1expression. Cytotherapy, 2012. 14(1): p. 70-9.

30. Liang, X. J., et al., Differentiation of human umbilical cord mesenchymal stem cells into hepatocyte-like cells by hTERT gene transfection in vitro. Cell Biol Int, 2012. 36(2): p. 215-21.

31. Fei, S., et al., The antitumor effect of mesenchymal stem cells transduced with a lentiviral vector expressing cytosine deaminase in a rat glioma model. J Cancer Res Clin Oncol, 2012. 138(2): p. 347-57.

32. Jaganathan, B. G. and D. Bonnet, Human mesenchymal stromal cells senesce with exogenous OCT4. Cytotherapy, 2012. 14(9): p. 1054-63.

33. Han, S. H., et al., Effect of ectopic OCT4 expression on canine adipose tissue-derived mesenchymal stem cell proliferation. Cell Biol Int, 2014. 38(10): p. 1163-73.

34. Shangguan, L., et al., Inhibition of TGF-beta/Smad signaling by BAMBI blocks differentiation of human mesenchymal stem cells to carcinoma-associated fibroblasts and abolishes their protumor effects. Stem Cells, 2012. 30(12): p. 2810-9.

35. Kearns-Jonker, M., et al., Genetically Engineered Mesenchymal Stem Cells Influence Gene Expression in Donor Cardiomyocytes and the Recipient Heart. J Stem Cell Res Ther, 2012. S1.

36. Ma, G. L., et al., [Study of inhibiting and killing effects of transgenic LIGHT human umbilical cord blood mesenchymal stem cells on stomach cancer]. Zhonghua Wei Chang Wal Ke Za Zhi, 2012. 15(11): p. 1178-81.

37. Huang, F., et al., Mesenchymal stem cells modified with miR-126 release angiogenic factors and activate Notch ligand Delta-like-4, enhancing ischemic angiogenesis and cell survival. Int J Mol Med, 2013. 31(2): p. 484-92.

38. Huang, F., et al., Overexpression of miR-126 promotes the differentiation of mesenchymal stem cells toward endothelial cells via activation of PI3K/Akt and MAPK/ERK pathways and release of paracrine factors. Biol Chem, 2013. 394(9): p. 1223-33.

39. Fang, Z., et al., Differentiation of GFP-Bcl-2-engineered mesenchymal stem cells towards a nucleus pulposus-like phenotype under hypoxia in vitro. Biochem Biophys Res Commun, 2013. 432(3): p. 444-50.

40. Madonna, R., et al., Transplantation of mesenchymal cells rejuvenated by the overexpression of telomerase and myocardin promotes revascularization and tissue repair in a murine model of hindlimb ischemia. Circ Res, 2013. 113(7): p. 902-14.

41. Zang, Y., et al., [Influence of CXCR4 overexpressed mesenchymal stem cells on hematopoietic recovery of irradiated mice]. Zhongguo Shi Yan Xue Ye Xue Za Zhi, 2013. 21(5): p. 1261-5.

42. Cao, Z., et al., Protective effects of mesenchymal stem cells with CXCR4 up-regulation in a rat renal transplantation model. PLoS One, 2013. 8(12): p. e82949.

43. Liu, S., et al., Overexpression of Wnt11 promotes chondrogenic differentiation of bone marrow-derived mesenchymal stem cells in synergism with TGF-beta. Mol Cell Biochem, 2014. 390(1-2): p. 123-31.

44. Zhu, Y., et al., Mesenchymal stem cell-based NK4 gene therapy in nude mice bearing gastric cancer xenografts. Drug Des Devel Ther, 2014. 8: p. 2449-62.

45. Yu, B., et al., Enhanced mesenchymal stem cell survival induced by GATA-4 overexpression is partially mediated by regulation of the miR-15 family. Int J Biochem Cell Biol, 2013. 45(12): p. 2724-35.

46. Xu, W., et al., Basic fibroblast growth factor expression is implicated in mesenchymal stem cells response to light-induced retinal injury. Cell Mol Neurobiol, 2013. 33(8): p. 1171-9.

47. Li, W., et al., Bcl-2 engineered MSCs inhibited apoptosis and improved heart function. Stem Cells, 2007. 25(8): p. 2118-27.

48. Tsubokawa, T., et al., Impact of anti-apoptotic and anti-oxidative effects of bone marrow mesenchymal stem cells with transient overexpression of heme oxygenase-1 on myocardial ischemia. Am J Physiol Heart Circ Physiol, 2010. 298(5): p. H1320-9.

49. Zhou, H., et al., Exendin-4 protects adipose-derived mesenchymal stem cells from apoptosis induced by hydrogen peroxide through the PI3K/Akt-Sfrp2 pathways. Free Radic Biol Med, 2014. 77: p. 363-75.

50. Le Blanc, K., et al., Treatment of severe acute graft-versus-host disease with third party haploidentical mesenchymal stem cells. Lancet, 2004. 363(9419): p. 1439-41.

51. Lazarus, H. M., et al., Cotransplantation of HLA-identical sibling culture-expanded mesenchymal stem cells and hematopoietic stem cells in hematologic malignancy patients. Biol Blood Marrow Transplant, 2005. 11(5): p. 389-98.

52. Bernardo, M. E., et al., Optimization of in vitro expansion of human multipotent mesenchymal stromal cells for cell-therapy approaches: further insights in the search for a fetal calf serum substitute. J Cell Physiol, 2007. 211(1): p. 121-30.

53. Reinisch, A., et al., Humanized system to propagate cord blood-derived multipotent mesenchymal stromal cells for clinical application. Regen Med, 2007. 2(4): p. 371-82.

54. Capelli, C., et al., Human platelet lysate allows expansion and clinical grade production of mesenchymal stromal cells from small samples of bone marrow aspirates or marrow filter washouts. Bone Marrow Transplant, 2007. 40(8): p. 785-91.

55. Lataillade, J. J., et al., New approach to radiation burn treatment by dosimetry-guided surgery combined with autologous mesenchymal stem cell therapy. Regen Med, 2007. 2(5): p. 785-94.

56. Seshareddy, K., D. Troyer, and M. L. Weiss, Method to isolate mesenchymal-like cells from Wharton's Jelly of umbilical cord. Methods Cell Biol, 2008. 86: p. 101-19.

57. Sensebe, L., Clinical grade production of mesenchymal stem cells. Biomed Mater Eng, 2008. 18(1 Suppl): p. S3-10.

58. Sotiropoulou, P. A., S. A. Perez, and M. Papamichail, Clinical grade expansion of human bone marrow mesenchymal stem cells. Methods Mol Biol, 2007. 407: p. 245-63.

59. Shetty, P., et al., Clinical grade mesenchymal stem cells transdifferentiated under xenofree conditions alleviates motor deficiencies in a rat model of Parkinson's disease. Cell Biol Int, 2009. 33(8): p. 830-8.

60. Zhang, X., et al., Cotransplantation of HLA-identical mesenchymal stem cells and hematopoietic stem cells in Chinese patients with hematologic diseases. Int J Lab Hematol, 2010. 32(2): p. 256-64.

61. Arrigoni, E., et al., Isolation, characterization and osteogenic differentiation of adipose-derived stem cells: from small to large animal models. Cell Tissue Res, 2009. 338(3): p. 401-11.

62. Grisendi, G., et al., GMP-manufactured density gradient media for optimized mesenchymal stromal/stem cell isolation and expansion. Cytotherapy, 2010. 12(4): p. 466-77.

63. Prasad, V. K., et al., Efficacy and safety of ex vivo cultured adult human mesenchymal stem cells (Prochymal) in pediatric patients with severe refractory acute graft-versus-host disease in a compassionate use study. Biol Blood Marrow Transplant, 2011. 17(4): p. 534-41.

64. Sensebe, L., P. Bourin, and K. Tarte, Good manufacturing practices production of mesenchymal stem/stromal cells. Hum Gene Ther, 2011. 22(1): p. 19-26.

65. Capelli, C., et al., Minimally manipulated whole human umbilical cord is a rich source of clinical-grade human mesenchymal stromal cells expanded in human platelet lysate. Cytotherapy, 2011. 13(7): p. 786-801.

66. Ilic, N., et al., Manufacture of clinical grade human placenta-derived multipotent mesenchymal stromal cells. Methods Mol Biol, 2011. 698: p. 89-106.

67. Santos, F., et al., Toward a clinical-grade expansion of mesenchymal stem cells from human sources: a microcarrier-based culture system under xeno-free conditions. Tissue Eng Part C Methods, 2011. 17(12): p. 1201-10.

68. Timmins, N. E., et al., Closed system isolation and scalable expansion of human placental mesenchymal stem cells. Biotechnol Bioeng, 2012. 109(7): p. 1817-26.

69. Warnke, P. H., et al., A clinically-feasible protocol for using human platelet lysate and mesenchymal stem cells in regenerative therapies. J Craniomaxillofac Surg, 2013. 41(2): p. 153-61.

70. Fekete, N., et al., GMP-compliant isolation and large-scale expansion of bone marrow-derived MSC. PLoS One, 2012. 7(8): p. e43255.

71. Hanley, P. J., et al., Manufacturing mesenchymal stromal cells for phase I clinical trials. Cytotherapy, 2013. 15(4): p. 416-22.

72. Trojahn Kolle, S. F., et al., Pooled human platelet lysate versus fetal bovine serum-investigating the proliferation rate, chromosome stability and angiogenic potential of human adipose tissue-derived stem cells intended for clinical use. Cytotherapy, 2013. 15(9): p. 1086-97.

73. Veronesi, E., et al., Transportation conditions for prompt use of ex vivo expanded and freshly harvested clinical-grade bone marrow mesenchymal stromal/stem cells for bone regeneration. Tissue Eng Part C Methods, 2014. 20(3): p. 239-51.

74. Dolley-Sonneville, P. J., L. E. Romeo, and Z. K. Melkoumian, Synthetic surface for expansion of human mesenchymal stem cells in xeno-free, chemically defined culture conditions. PLoS One, 2013. 8(8): p. e70263.

75. Siciliano, C., et al., Optimization of the isolation and expansion method of human mediastinal-adipose tissue derived mesenchymal stem cells with virally inactivated GMP-grade platelet lysate. Cytotechnology, 2015. 67(1): p. 165-74.

76. Martins, J. P., et al., Towards an advanced therapy medicinal product based on mesenchymal stromal cells isolated from the umbilical cord tissue: quality and safety data. Stem Cell Res Ther, 2014. 5(1): p. 9.

77. Iudicone, P., et al., Pathogen-free, plasma-poor platelet lysate and expansion of human mesenchymal stem cells. J Transl Med, 2014. 12: p. 28.

78. Skrahin, A., et al., Autologous mesenchymal stromal cell infusion as adjunct treatment in patients with multidrug and extensively drug-resistant tuberculosis: an open-label phase 1 safety trial. Lancet Respir Med, 2014. 2(2): p. 108-22.

79. Ikebe, C. and K. Suzuki, Mesenchymal stem cells for regenerative therapy: optimization of cell preparation protocols. Biomed Res Int, 2014. 2014: p. 951512.

80. Chatzistamatiou, T. K., et al., Optimizing isolation culture and freezing methods to preserve Wharton's jelly's mesenchymal stem cell (MSC) properties: an MSC banking protocol validation for the Hellenic Cord Blood Bank. Transfusion, 2014. 54(12): p. 3108-20.

81. Swamynathan, P., et al., Are serum free and xeno-free culture conditions ideal for large scale clinical grade expansion of Wharton's jelly derived mesenchymal stem cells? A comparative study. Stem Cell Res Ther, 2014. 5(4): p. 88.

82. Vaes, B., et al., Culturing protocols for human multipotent adult stem cells. Methods Mol Biol, 2015. 1235: p. 49-58.

83. Devito, L., et al., Wharton's jelly mesenchymal stromal/stem cells derived under chemically defined animal product-free low oxygen conditions are rich in MSCA-1(+) subpopulation. Regen Med, 2014. 9(6): p. 723-32.

1. A method of generating mesenchymal stem cells with enhanced therapeutic activity, said method comprising treatment of mesenchymal stem cells with one or more Noble gases at a concentration and frequency sufficient to induce more than 5% increase in HIF-1 alpha expression in said mesenchymal stem cell.

2. The method of claim 1, wherein said mesenchymal stem cells are selected from a group comprising of: a) bone marrow; b) perivascular tissue; c) adipose tissue; d) placental tissue; e) amniotic membrane; f) omentum; g) tooth; h) umbilical cord tissue; i) fallopian tube tissue; j) hepatic tissue; k) renal tissue; l) cardiac tissue; m) tonsillar tissue; n) testicular tissue; o) ovarian tissue; p) neuronal tissue; q) auricular tissue; r) colonic tissue; s) submucosal tissue; t) hair follicle tissue; u) pancreatic tissue; v) skeletal muscle tissue; and w) subepithelial umbilical cord tissue.

3. The method of claim 1, wherein said mesenchymal stem cells express a marker selected from a group comprising of: a) CD73; b) CD90; and c) CD105.

4. The method of claim 1, wherein said mesenchymal stem cells lack expression of a marker selected from a group comprising of: a) CD14; b) CD45; and c) CD34.

5. The method of claim 1, wherein said efficacy reflects enhanced ability to induce clinical response in a disease condition.

6. The method of claim 6, wherein said disease condition is selected from a group comprising of: a) neurological disease; b) inflammatory conditions; c) psychiatric disorders; d) inborn errors of metabolisms; e) vascular disease; f) cardiac disease; g) renal disease; h) hepatic disease; i) pulmonary disease; j) ocular conditions; k) gastrointestinal disorders; l) orthopedic disorders; m) dermal disorders; n) neoplasia; o) predisposition to neoplasia; p) hematopoietic disorders; q) reproductive disorders; r) gynecological disorders; s) urological disorders; t) immunological disorders; u) olfactory disorders; and v) auricular disorders.

7. The method of claim 1, wherein said mesenchymal stem cells selected for enhanced efficacy are utilized as a source of conditioned media.

8. The method of claim 7, wherein conditioned media is used therapeutically in the treatment of a disorder.

9. The method of claim 8, wherein said disorder is selected from a group comprising of a) neurological disease; b) inflammatory conditions; c) psychiatric disorders; d) inborn errors of metabolisms; e) vascular disease; f) cardiac disease; g) renal disease; h) hepatic disease; i) pulmonary disease; j) ocular conditions; k) gastrointestinal disorders; l) orthopedic disorders; m) dermal disorders; n) neoplasia; o) predisposition to neoplasia; p) hematopoietic disorders; q) reproductive disorders; r) gynecological disorders; s) urological disorders; t) immunological disorders; u) olfactory disorders; and v) auricular disorders.

10. The method of claim 1, wherein said Noble gas is xenon.

11. The method of claim 1, wherein said Noble gas is a mixture, said mixture containing one or more of the following gases: a) nitrogen; b) helium; c) Nitric Oxide; d) krypton; e) argon; and f) neon.

12. The method of claim 1, wherein said mesenchymal stem cells are treated with said Noble gas containing mixture by incubating said cells in an atmosphere containing said gases.

13. The method of claim 1, wherein said Noble gas mixture containing said Noble gas is administered into culture media by dissolving of said Noble gas or mixture containing said Noble gas into said culture media.

14. The method of claim 1, wherein said noble gas containing mixture is comprised of a gas mixture containing oxygen and a proportion by volume of 20 to 70% of xenon.

15. The method of claim 65, wherein said proportion of xenon is between 22 and 60% by volume to oxygen.

16. The method of claim 65, wherein said proportion of xenon is between 25 and 60% by volume to oxygen.

17. The method of claim 1, wherein said noble gas containing mixture consists only of a) oxygen and xenon or b) air and xenon.

18. The method of claim 1, wherein said noble gas containing mixture also contains nitrogen, helium, Nitric Oxide, krypton, argon or neon.

19. The method of claim 1, wherein said noble gas containing mixture contains a proportion by volume of oxygen of between 15 and 25%.
